(19) United States
(12) Patent Application Publication
(10) Pub. No.: US 2007/0040553 A1
(43) Pub. Date: Feb. 22, 2007

(54) Spectral-scanning magnetic resonance imaging

(76) Inventors: Seyed Ali Hajimiri, Pasadena, CA (US); Arjang Hassibi, Pasadena, CA (US); Hua Wang, Pasadena, CA (US)

Correspondence Address: GREER, BURNS & CRAIN, 300 S WACKER DR, 25TH FLOOR, CHICAGO, IL, 60606, US

(21) Appl. No.: 11/499,921

(22) Filed: Aug. 7, 2006

Related U.S. Application Data
(60) Provisional application No. 60/706,406, filed on Aug. 8, 2005.

(51) Int. Cl. G01V 3/00; A61B 5/05

(52) U.S. Cl. 324/309; 600/410

(57) ABSTRACT

Spectral scanning magnetic resonance imaging methods and systems. In preferred methods and systems of the invention, to measure the resonance spectrum of the target object, a plurality of excitation signals in different frequencies and/or waveform shapes are introduced simultaneously to the imaging volume through one or more excitation coils, and the response spectrum is measured also in real-time and/or after excitation. Systems of the invention can be compact and portable, with small magnets providing the deterministic inhomogeneous magnetic field. Preferred embodiments include integrated circuit transmitters and receivers. Preferred systems of the invention are suitable, for example, for point of care medical diagnostics.

### CLAIM FOR PRIORITY AND REFERENCE TO RELATED APPLICATION

This application claims priority under 35 U.S.C. §119 to prior provisional application 60/706,406 filed Aug. 8, 2005.

### FIELD OF THE INVENTION

A field of the invention is the field of magnetic resonance. Example applications of the invention include, but are not limited to, microscopic resonance imaging, spectrometry, and general resonance imaging.

### BACKGROUND

Magnetic Resonance Imaging (MRI) is an imaging technique used primarily in medical settings to produce images of the inside of biologically-relevant objects such as the human body. MRI is based on the principles of nuclear magnetic resonance (NMR); a spectroscopic technique used to obtain chemical and physical information about molecules and chemical bonds. In a typical MRI imaging device, a large direct current magnetic field is applied and a perpendicular alternating current magnetic field is applied for excitation. The alternating current creates a field that permits resonant spins to be detected in the presence of other spins. Resonance imaging is based upon the fact that images can be calculated from the detected resonance spins.

In conventional MRI platforms, the excitation signal is narrow-band (i.e., the bandwidth of the signal is much smaller than the carrier RF frequency), where the RF center frequency is adjusted to be the resonance frequency of hydrogen nuclei at the selected imaging coordinate. With conventional techniques, accurate MRI requires a very strong, yet controlled level of magnetization within the object being imaged. Creating the strong, uniform magnetic field is a fundamental challenge of MRI imaging, which limits its applications.

Most MRI platforms implement magnets which are of the superconducting type to generate the required strong magnetic field. By utilizing correction coils with the superconductor magnet, the setup generates the required controlled and uniform magnetization within the object. Typical MRI applications necessitate uniformity in the order of one part per million (1 ppm) for the magnetic field. Nevertheless, this magnetic field is adjustable by superimposing additional magnetic field gradients, generated by gradient coils which can be turned on and off rapidly. Activation of these additional magnetic fields results in a net gradient in the strength of the magnetic field across the object, which is essential for spatial localization and imaging. Such approaches are highly practical, but make the magnetization apparatus the most expensive, bulky, and perhaps complicated component of conventional MRI imaging systems.

Modem techniques for MRI imaging include more than hydrogen nuclei density 3-D imaging. Similar magnetic resonance-based imaging techniques using existing MRI device/magnetization platforms have been developed. Examples of such techniques are flow imaging (MRI angiography), diffusion imaging, chemical shift imaging (fat suppression), T1 and T2 density imaging, hyperpolarized noble gas imaging, and parallel imaging. These techniques have different strengths and weaknesses, but all share the common practical drawback of conventional MRI, which is the bulkiness and complexity of the magnetization setup due to the required uniformity of the magnets. This consequently limits the MRI imaging methods to applications where a stationary imaging platform can be used.

With conventional MRI imaging systems, reducing the magnetic field generation platform (including the magnet(s)) would introduce a high level of nonuniformity within the magnetic field. This is an inherent result of isomorphic scaling (i.e., scaling in all dimensions). The nonuniformity introduces drastic degradation of the signal-to-noise ratio (SNR) and signal relaxation time, which is why conventional systems continue to use large magnetic field generation platforms despite the cost and inconvenience that they introduce into conventional MRI imaging systems.

### SUMMARY OF THE INVENTION

The invention provides spectral scanning magnetic resonance imaging methods and systems. In preferred methods and systems of the invention, to measure the resonance spectrum of the target object, a plurality of excitation signals in different frequencies and/or waveform shapes are introduced simultaneously to the imaging volume through one or more excitation coils, and the response spectrum is measured also in real-time and/or after excitation.

### BRIEF DESCRIPTION OF THE DRAWINGS

FIG. 1 schematically illustrates a preferred embodiment spectral scanning magnetic field generation system of the invention;

FIG. 2A-2C illustrate a method for generating an alternative magnetic resonance response matrix by moving the location of the object within the magnetization field;

FIGS. 3A-3C illustrate a method for generating an alternative magnetic resonance response matrix with correction coils;

FIG. 4 is a block diagram of a preferred embodiment spectral scanning magnetic resonance imaging system of the invention;

FIG. 5 is a block diagram of a preferred embodiment integrated transmitter architecture for generating spectral scanning magnetic resonance imaging frequencies according to the invention;

FIG. 6 is a block diagram of another preferred embodiment integrated transmitter architecture for generating spectral scan magnetic resonance imaging frequencies according to the invention;

FIG. 7 is a block diagram of a preferred embodiment digital signal generator for an integrated transmitter architecture such as the FIGS. 5 and 6 architectures;

FIG. 8 is a block diagram of a preferred embodiment digital I and Q generator for an integrated transmitter architecture such as the FIG. 6 architecture; and

FIG. 9 illustrates a preferred embodiment direct conversion architecture for a spectral scanning magnetic resonance imaging receiver of the invention.

### DETAILED DESCRIPTION OF PREFERRED EMBODIMENTS

The invention provides spectral scanning magnetic resonance imaging methods and systems. In preferred methods and systems of the invention, to measure the resonance spectrum of the target object, a plurality of excitation signals in different frequencies and/or waveform shapes are introduced simultaneously to the imaging volume through one or more excitation coils, and the response spectrum is measured also in real-time and/or after excitation. Imaging methods of the invention are referred to as spectral scanning magnetic resonance imaging (SSMRI). The SSMRI analysis can be conducted in real-time.

In example embodiments, SSMRI integrated circuit systems and/or system-on-a-chip (SoC) platforms are provided, and are capable of simultaneously generating a broad-band excitation signal and detecting the object response spectrum. An important application of SSMRI systems is in tomography, in particular medical imaging. An advantage of SSMRI over conventional MRI is that the device size can be substantially reduced, permitting use, for example, in point-of-care (PoC) medical diagnostic, where instrument portability, magnet size, imaging speed, and versatility are imperative.

Embodiments of the invention greatly reduce the burdens associated with uniformity of magnetic field that are present in conventional devices for magnetic resonance imaging. Preferred MRI imaging devices of the invention can be portable. A portable MRI imaging device of the invention is versatile, and can be applied to applications such as point-of-care (PoC) medical diagnostics. Preferred devices of the invention include scaled down magnetic field generation platforms, permitting the downsizing of the entire MRI imaging apparatus, while sensitivity is maintained within the requirements of the conventional MRI imaging systems.

In SSMRI methods and systems of the invention, the target object (i.e., the object to be imaged by magnetic resonance tomography) is placed within a controlled and deterministic inhomogeneous (non-uniform) magnetic field created by a magnetic field generation system including one and/or a plurality of permanent magnets and/or magnetic coils and/or superconducting magnets. Since the strength of the magnetic field \vec{B}₀(x,y,z), is non-uniform within the imaging volume (i.e., the defined volume in which the SSMRI system carries out magnetic resonance tomography), the magnetic resonance frequency ω_{R} of identical nuclei spins (e.g., H¹ or C¹²) of the target become coordinate-dependant, such that ω_{R} ∝|B₀(x,y,z)|. Hence, different coordinates within the target object will resonate at different resonance frequencies. By measuring the resonance spectrum (e.g., a plurality of defined sinusoidal tones) within the possible resonance frequencies present, the imaging volume for particular nuclei, it is possible to assess information regarding the spatial density of those nuclei, and therefore assess tomographic information related with the target.

In certain embodiments of the invention, to measure the resonance spectrum of the target object, a plurality of excitation signals in different frequencies and/or waveform shapes are introduced simultaneously to the imaging volume through one or more excitation coils, and the response spectrum is measured also in real-time and/or after excitation. The response is measured using one or more receiving coils and/or Hall-effect sensors and/or superconducting quantum interference devices (SQUID) connected to the sensor and data acquisition apparatus.

In other embodiments of the invention, one or more excitation signals in different frequencies -and/or waveform shapes are modified as a function of time. The spectral response is also measured and analyzed as a function of time, following the excitation signal changes.

In other embodiments of the invention, the spatial magnetic field within the target object is altered by using one or more additional field-adjustment magnetic coils and/or movement of the object with respect to the original field. The additional tomographic information from one or more modified field measurements along with the original measurement can be used to construct a more detailed tomographic image of the target object.

In certain embodiments of the invention, to generate and measure the resonance spectrum, an integrated semiconductor-based integrated chip is used. In this case, the excitation spectrum and/or waveforms are generated by the circuitry within the integrated circuit and put onto the external and/or integrated excitation coils. The integrated and/or receiving coils are connected within sensor circuitry fabricated within the chip.

Additional preferred embodiments of the invention will now be discussed with reference to the drawings. Artisans will recognize broader aspects of the invention from the following discussion of preferred embodiments and the above calculations and principles. Additional embodiments of the invention will also be apparent to artisans from the following discussion. The preferred embodiments will be discussed with respect to a preferred magnetic resonance imaging application, but artisans will also understand the broader applicability of the invention.

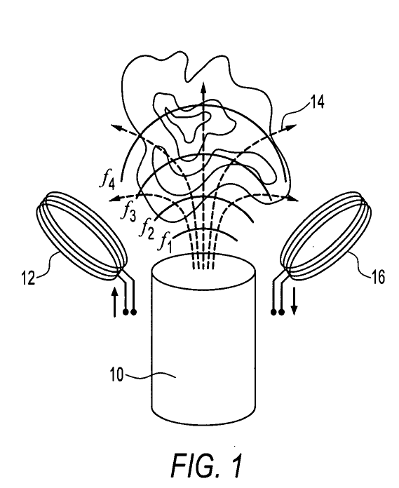

FIG. 1 shows a preferred embodiment spectral scanning magnetic resonance generation system of the invention. A magnet 10 generates an inhomogeneous magnetic field. The magnet 10 may be a single permanent magnet, for example, or can be a plurality of permanent magnets. A controlled and deterministic inhomogeneous (non-uniform) magnetic field is created by the magnet 10, which can also be realized, for example, by one and/or a plurality of permanent magnets and/or magnetic coils and/or superconducting magnets.

An excitation coil 12 provides an excitation signal to create resonance at different frequencies, e.g., f₁-f₄, within an imaging volume. Field lines 14 are intersected by arcs that indicate the equi-magnetic surfaces at the indicated frequencies f₁-f₄ in the imaging volume. A magnetic resonance sensor 16, e.g., a coil, receives the resonance spectrum. Removed from the constraint of uniformity, the magnet 10 can be compact, sized to create a portable imaging device, for example. The excitation coil 12 provides a plurality of excitation signals in different frequencies and/or waveform shapes simultaneously to the imaging volume through one or more excitation coils.

Example embodiments will be illustrated with multiple frequency excitation and detection. Artisans will appreciate that multiple waveform shape excitation can be used to produce information necessary for sensing a response in a target image and for analyzing the sensed signals to produce tomographic data.

The strength of the magnetic field \vec{B}₀(x,y,z), is non-uniform within the imaging volume (i.e., the defined volume in which the SSMRI system carries out magnetic resonance tomography), so that the magnetic resonance frequency ω_{R} of identical nuclei spins (e.g., H¹ or C¹²) of a target 18 become coordinate-dependant, such that ω_{R} ∝|B₀ (x, y, z)|. Hence, different coordinates within the target object will resonate at different resonance frequencies. By measuring the resonance spectrum (e.g., a plurality of defined sinusoidal tones) received by the magnetic resonance sensor 16 within the possible resonance frequencies present, the imaging volume for particular nuclei, it is possible to assess information for the spatial density of those nuclei, and therefore assess tomographic information related with the target.

The excitation coil 12 in preferred embodiments is realized by a plurality of excitation coils. In preferred embodiments, a plurality of excitation coils 12 generate a plurality of excitation signals in different frequencies and/or waveform shapes, which are introduced simultaneously to the imaging volume. The magnetic resonance sensor 16 preferably measures the response spectrum in real-time. The sensor 16 can be realized by one or more sensor coils, and/or Hall-effect sensors, and/or superconducting quantum interference devices (SQUID). In preferred imaging systems of the invention the sensor's output is provided to a data acquisition apparatus.

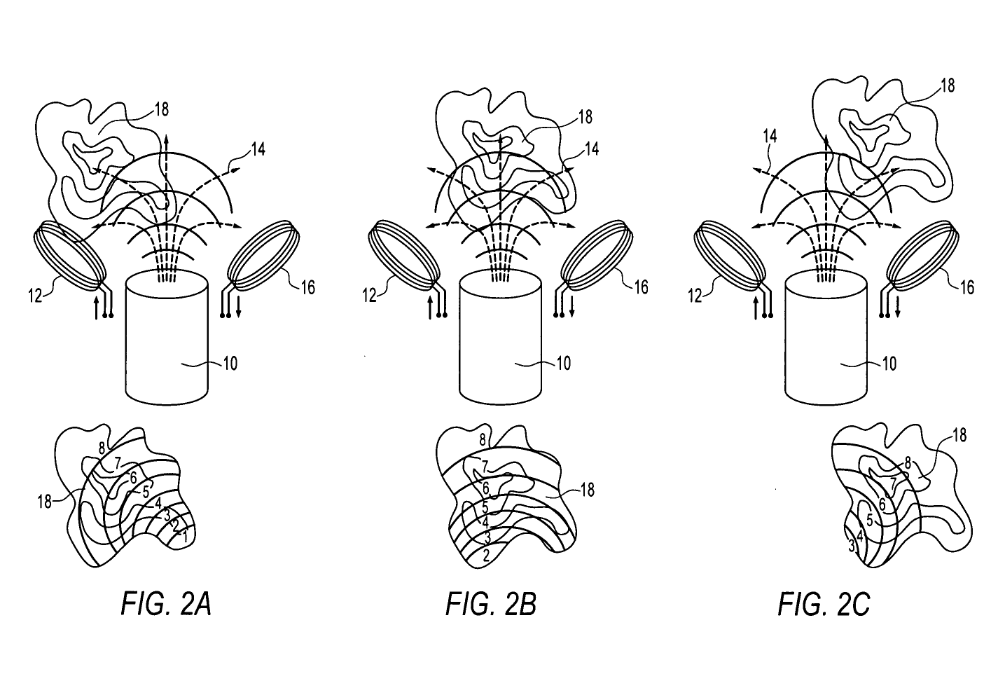

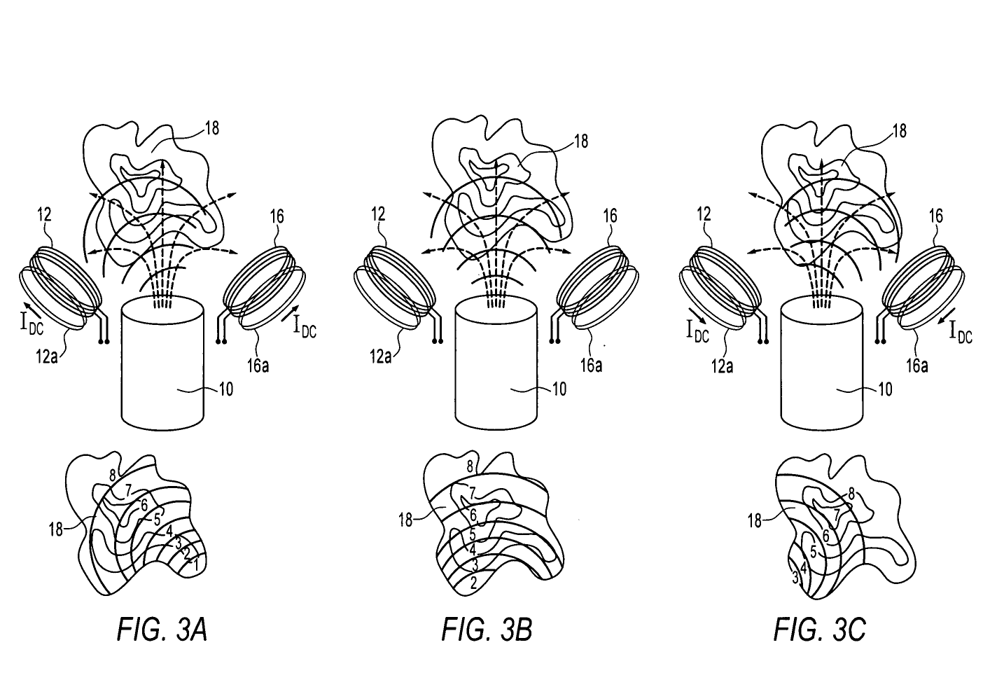

FIG. 2A-2C illustrate a method for generating an alternative magnetic resonance response matrix by moving the location of the object within the magnetization field. FIGS. 3A-3C illustrate a method for generating an alternative magnetic resonance response matrix with correction coils. Reference numbers from FIG. 1 have been used to identify comparable parts in FIGS. 2A-2C, and in FIGS. 3A-3C.

FIGS. 2A-2C respectively show 3 separate locations for the target object 18 being imaged. By moving the location of the target object 18 within the magnetization field, a new set of frequency spectrum data points can be extracted for imaging. The numbers 1 to 8 correspond to coordinates in the target object 18 which share a common resonance frequency (equi-magnetic field surfaces).

FIGS. 3A-3C show an alternate method for generating a new set of frequency spectrum data points that permit the target object 18 being imaged to remain stationary. In addition to excitation coil 12 and receiving coil 16, the spectral scanning magnetic field generation system of FIGS. 3A-3C includes one or more excitation correction coils 12a and sensor correction coils 16a. By changing the magnetization field density using the correction coils 12a, 16a, a new set of frequency spectrum data points can be extracted for imaging. The numbers 1 to 8 correspond to coordinates in the target object which share a common resonance frequency (equi-magnetic field surfaces). The correction coils 12a and 16a are provided with a correction current I_{DC} in FIG. 3A. In FIG. 3B, no current is supplied to the correction coils 12a and 16a. In FIG. 3C, the correction current −I_{DC} is provided in the correction coils 12a and 16a. The correction currents or lack thereof provide modified magnetic excitation signals in the imaging volume. These are three exemplary cases, and artisans will appreciate that variations in current levels can produce many different sets of frequency spectrum data points.

The spectral scanning magnetic resonance systems of FIGS. 1-3 induce a resonance in the target object 18 that can be sensed by the sensor 16, e.g. receiving coils, and analyzed to construct an image. Characterization of the induced magnetization provides an example method for image reconstruction.

Consider that the target object is subject to an initial inhomogeneous magnetic field \vec{B}₀(x,y,z), and given the excitation signals in time, a magnetization vector \vec{M}(x,y,z,t) as a function of time. If the normalized magnetic field of the k^{th} receiving coil at the coordinate r=(x, y, z) is \vec{B}_{N}^{(k)}(r), then the induced magnetic current at the k^{th} receiving coil (of sensor 16) from the nuclei spins at frequency is defined by

$$d ɛ ( t ) = - \frac{\partial }{\partial t} [ {M}^{->} ( r ) \cdot {{B}^{->}}_{N}^{( k )} ( r ) ] dV , \qquad ( 1 )$$

and accordingly for a small volume ΔV around r₀=(x₀, y₀,z₀) one can rewrite (1) as

$${ɛ}^{( k )} ( t , {r}_{0} ) = - \frac{\partial }{\partial t} [ {M}^{->} ( {r}_{0} ) \cdot {{B}^{->}}_{N}^{( k )} ( {r}_{0} ) ] Δ V . \qquad ( 2 )$$

It can be shown that the induced signal ε^{(k)}(t,r₀) is a narrowband signal where its center frequency is located around ω(r₀) the resonance frequency of the nuclei spins at r₀. If the gyromagnetic ratio is γ, then this frequency is ω(r₀)=γ|\vec{B}₀(r₀)|. It can also be shown that magnitude of ε^{(k)}(t, r₀) is proportional to nuclei spin density at r₀, defined by ρ(r₀), such that
ε^{(k)}(t,r₀)=F^{(k)}(r₀,t)ρ(r₀)ΔV, (3)

where function F^{(k)}(r,t) is the magnetic resonance response function, describing the induced signal of the spins at r₀ into the k^{th} coil of the sensor 16. This function is deterministic and is independent of the object tomographic information described by ρ.

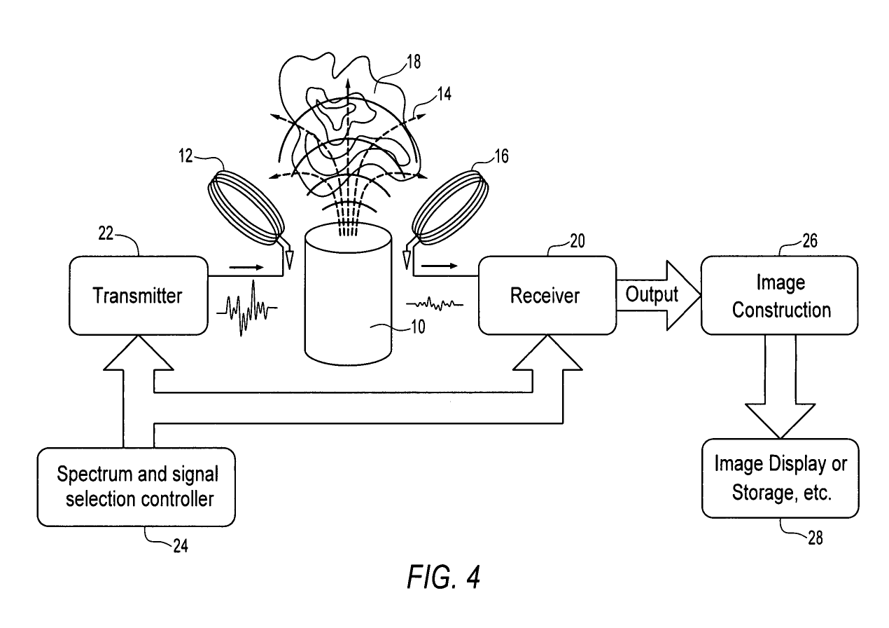

FIG. 4 is a block diagram of a preferred embodiment spectral scanning magnetic resonance imaging system of the invention. The SSMRI imaging system includes a magnetic generation subsystem in accordance, for example, with the preferred embodiment of FIG. 1 and reference numbers from FIG. 1 are utilized to label comparable parts of the FIG. 4 system. The sensor 16 will sense the resonance response defined by (3), which is provided to a receiver 20. The excitation coil 12 receives signals from a transmitter 22, and the transmitter 22 and receiver 20 are controlled by a spectrum and signal selection controller 24 such that the receiver 20 can detect the object response spectrum.

The excitation spectrum is generated by the transmitter 22 and the receiver 20 measures the target object 18 response under control of the spectrum and signal selection controller 24. The output of the receiver 20 is provided to an image construction module 26, e.g., a computer or software module, to extract the tomographic information about the target object 18 and preferably construct a tomographic image.

In preferred embodiments, the transmitter 22 generates the excitation spectrum and is controlled by a user of the SSMRI system, whereas the receiver 20, in real-time, measures the response of the excitation spectrum by identifying and/or selectively amplifying and/or down-converting, and/or digitizing the response from other interfering signals and/or noise.

The image construction module 26 subsequently extracts tomographic information by analyzing the output of the receiver 20. The image construction module 26 can provide data to a display or storage module 28, for example.

In preferred embodiments, the analysis of the output of the receiver 20 can be considered as finding the value of ρ for n finite number of coordinates, r₀, r₁, . . . , rₙ₋₁, with volumes of ΔV₀, ΔV₁, . . . , ΔVₙ₋₁. First, it is assumed that the response of the system is observed by the receiver 20 in m different frequencies ω₀, ω₁, . . . , ωₘ₋₁, each having an induced signal Eᵢ^{(k)}, generated by the target object 18 at (in preferred embodiments where the sensor 16 is realized with sensor coils) sensor coil k at frequency ωᵢ. One can write Eᵢ^{(k)} as a function of ρ using the following summation:

$${E}_{i}^{( k )} = {\sum }_{j = 1}^{n} {f}_{i , j}^{( k )} {ρ}_{j} . \qquad ( 4 )$$

The function f_{i,j}^{(k)} basically is very similar to F^{(k)}(rⱼ,t) yet it also includes the volume ΔVⱼ and the frequency of operation such that

$${f}_{i , j}^{( k )} = \{ \begin{matrix}{F}^{( k )} ( {r}_{j} , t ) Δ {V}_{j} & if {ω}_{i} - δ \leq y \vert {B}_{0} ( {r}_{j} ) \vert \leq {ω}_{i} + δ \\ 0 & else\end{matrix} \qquad ( 5 )$$

where 2δ is the frequency bandwidth of ε^{(k)}(rⱼ,t), given the excitation waveform.

It is imperative to understand that f_{i,j}^{(k)} is a deterministic function and also independent of function ρⱼ. Hence, by employing function f_{i,j}^{(k)} as a scalar which relates ρⱼ to Eᵢ^{(k)}, the following linear system is defined

$$\begin{matrix}( \begin{matrix}{E}_{1}^{( k )} \\ {E}_{2}^{( k )} \\ {E}_{3}^{( k )} \\ ⋮ \\ {E}_{m}^{( k )}\end{matrix} ) = ( \begin{matrix}{f}_{1 , 1}^{( k )} & {f}_{1 , 2}^{( k )} & {f}_{1 , 3}^{( k )} & & {f}_{1 , m}^{( k )} \\ {f}_{2 , 1}^{( k )} & {f}_{2 , 2}^{( k )} & {f}_{2 , 3}^{( k )} & ⋯ & {f}_{2 , m}^{( k )} \\ {f}_{3 , 1}^{( k )} & {f}_{3 , 2}^{( k )} & {f}_{3 , 3}^{( k )} & & \\ & ⋮ & & ⋰ & ⋮ \\ {f}_{m , 1}^{( k )} & {f}_{m , 2}^{( k )} & {f}_{m , 2}^{( k )} & ⋯ & {f}_{m , n}^{( k )}\end{matrix} ) ( \begin{matrix}{ρ}_{1} \\ {ρ}_{2} \\ {ρ}_{3} \\ ⋮ \\ {ρ}_{n}\end{matrix} ) , \\ or & ( 6 ) \\ {E}^{( k )} = {F}^{( k )} \cdot ρ , & ( 7 )\end{matrix}$$

where E^{(k)}εR^{m}, F^{(k)}εR^{m×n}, and ρεRⁿ.

To find for ρ, solve (7), given the measurement vector E^{(k)}. Criteria which makes this possible is that rank(F^{(k)})≧n. If this criteria is satisfied there is sufficient (or even redundant) information to assess ρ. In certain cases, rank(F^{(k)})<n and thus to construct a tomographic image it is necessary to have more independent measurements, as provided by the methods illustrated in FIGS. 2A-2C and 3A-3C. Such measurements basically have the same p, however they have a different F matrix. Since F describes the magnetic resonance response function, by changing its variables it is possible to generate the modified matrix F^{(l)}, where F^{(l)≠F}^{(k)}. This response function, for the same object, will create a new set of measurement results E^{(l)}, where E^{(l)}=F^{(l)}·ρ. Data can be combined as

$$( \begin{matrix}{E}^{( k )} \\ {E}^{( l )}\end{matrix} ) = ( \begin{matrix}{F}^{( k )} \\ {F}^{( l )}\end{matrix} ) \cdot ρ , \qquad ( 8 )$$

with a more relaxed criteria of rank(F^{(k)},F^{(l)})≧n, where (F^{(k)},F^{(l)})εR^{2m×n}. For ρ set of experiments E¹), E⁽²⁾, . . . , E^{(ρ)} with magnetic resonance responses of F⁽¹⁾, F⁽²⁾, . . . , F^{(ρ)} it is possible to generalize (8) as

$$( \begin{matrix}{E}^{( 1 )} \\ {E}^{( 2 )} \\ ⋮ \\ {E}^{( p )}\end{matrix} ) = ( \begin{matrix}{F}^{( 1 )} \\ {F}^{( 2 )} \\ ⋮ \\ {F}^{( p )}\end{matrix} ) \cdot ρ , \qquad ( 9 )$$

with a general imaging criteria of rank(F⁽¹⁾, F⁽²⁾, . . . , F^{(ρ)})≧n.

In certain embodiments of SSMRI platform, integrated circuits are used to realize one or both of the transmitter 22 and/or receiver 20. Such systems can be realized in CMOS, BiCMOS, or Bipolar semiconductor fabrication processes. In some embodiments the image construction module 26 is realized with embedded digital signal processing (DSP) semiconductor chips.

In additional preferred embodiments the excitation coil 12 and the sensor 16, such as a sensor coil, are fabricated within the transmitter and receiver semiconductor chip. For example, integrated spiral inductor arrays connected to the receiver 20 circuitry on the same semiconductor chip can be used in the receiver 20 to sense the response of the target object 18 to the excitation signal.

In certain embodiments of the system, the spectrum and signal selection controller 24 is also realized with an integrated semiconductor-based integrated chip. In this case, the excitation spectrum and/or waveforms are generated by the circuitry within the integrated circuit and put onto the external and/or integrated excitation coils 12. In preferred embodiments, the excitation and/or receiving coils are also integrated within the chip. Because the magnet 10 required to generate the required inhomogeneous magnetic field can be compact, an entire SSMRI system of the invention can be compact and portable, suitable, for example, for point-of-care (PoC) medical diagnostics.

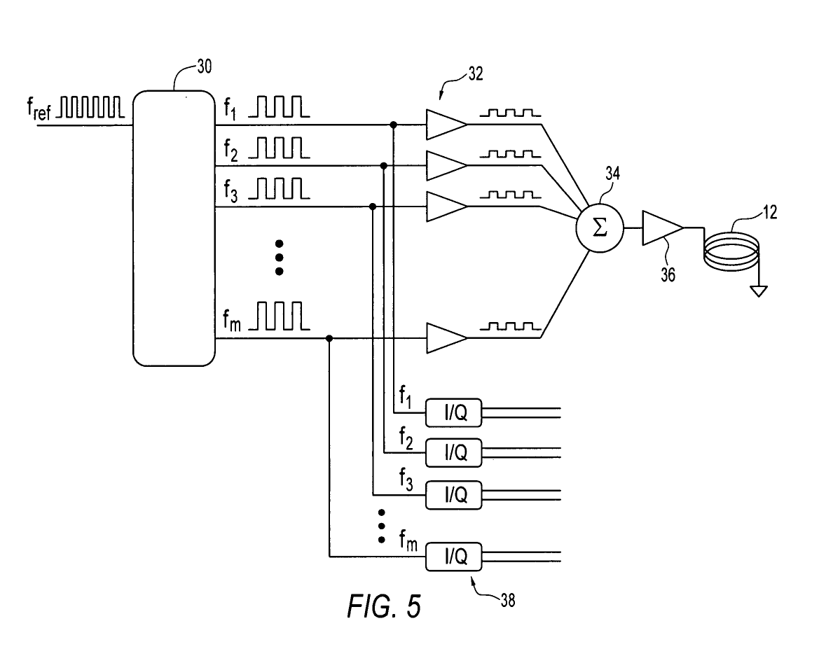

FIG. 5 is a block diagram of a preferred embodiment integrated transmitter architecture for generating spectral scanning magnetic resonance imaging frequencies, suitable as use for the transmitter 22 and spectrum signal selection controller 24 of the FIG. 4 SSMRI system. In FIG. 5, a digital signal generator 30 creates SSMRI frequencies using a frequency f_{ref}. The excitation spectrum is generated by a set of pulse-shaping amplifiers 32, whose outputs are summed by summer 34 and amplified by an output amplifier 36 and applied to the excitation coil 12. I and Q (i.e., 90° phase difference) signals are also generated in the transmitter 22 by I and Q generators 38. In other embodiments, envelope-shaping amplifiers may be used.

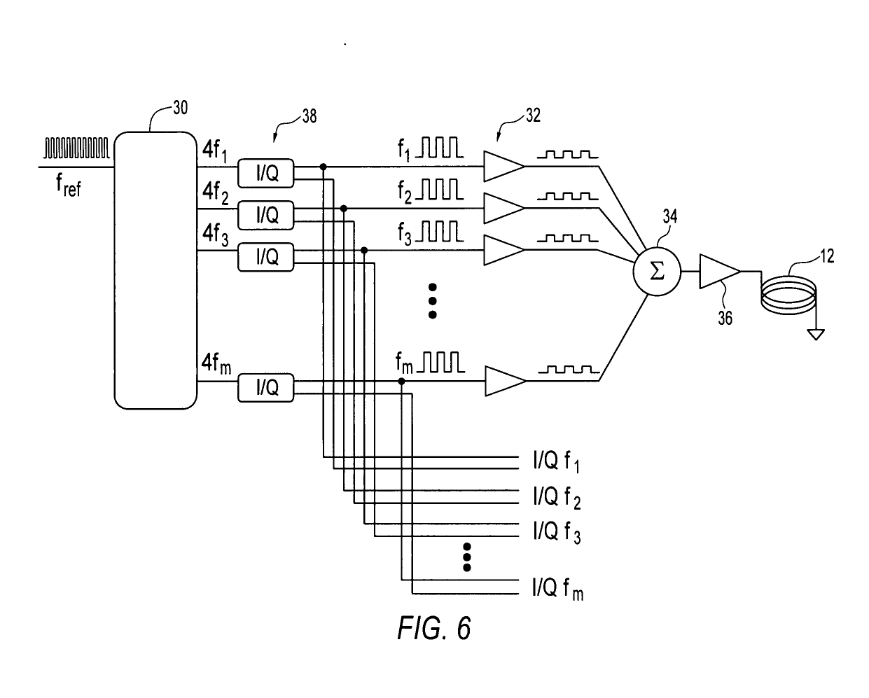

FIG. 6 shows another preferred embodiment integrated transmitter architecture for generating spectral scanning magnetic resonance imaging frequencies, suitable as use for the transmitter 22 and spectrum signal selection controller 24 of the FIG. 4 SSMRI system. Comparable parts of the FIG. 6 integrated transmitter are labeled with reference numbers from FIG. 6. In the FIG. 6 transmitter architecture, the digital signal generator 30 creates four times the SSMRI frequencies using the reference frequency f_{ref}. The excitation spectrum is generated by the set of pulse-shaping amplifiers 32 after creating the I and Q (90° phase difference) signals digitally.

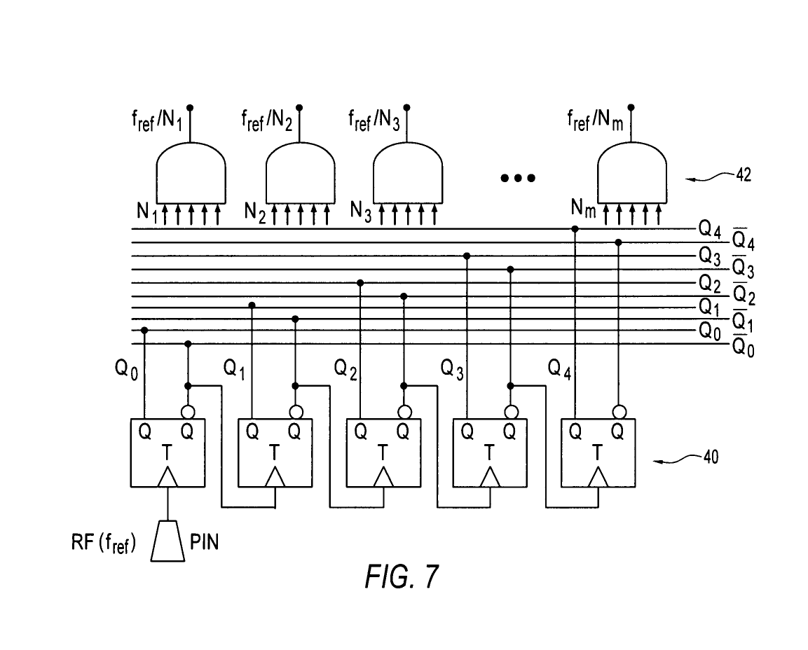

FIG. 7 is a block diagram of a preferred embodiment digital signal generator for an integrated transmitter architecture such as the FIGS. 5 and 6 architectures. In FIG. 7, the basic arrangement is that of a digital divider, with T-flip flops 40 receiving the reference frequency f_{ref} and connected as a ripple counter, with the SSMRI frequencies being provide from respective AND gates 42.

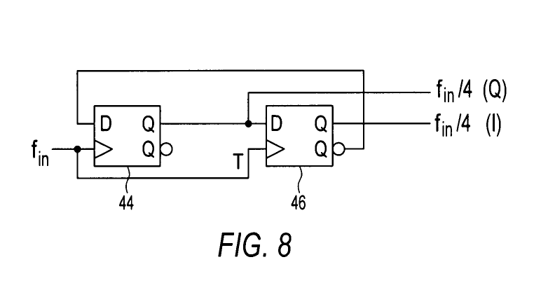

FIG. 8 is a block diagram of a preferred embodiment digital I and Q generator for an integrated transmitter architecture of FIG. 6. A first D flip-flop 44 generates a first phase, Q phase, from a respective frequency signal (f₁-fₘ) provided by the digital signal processor 30. A second D flip-flop 46 provides a delay to generate the 90 degree difference phase, I phase.

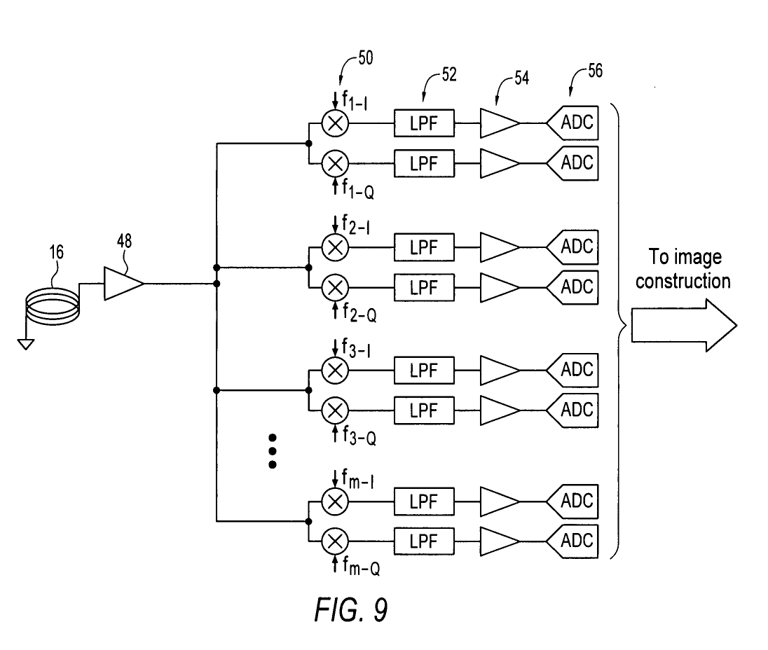

FIG. 9 illustrates a preferred embodiment direct conversion architecture for a spectral scanning magnetic resonance imaging receiver of the invention that is suitable as use for the receiver 20 of the FIG. 4 SSMRI system. The signal received by the sensor coil 16 is first amplified by a low noise amplifier 48, and then down-converted by I and Q signals for each frequency within the spectrum of interest by a mixer 50. Signals from the mixer 50 are filtered by a low pass filter 52, amplified by amplifiers 54, and digitized by an A/D converter 56. The I and Q (90° phase difference) signals, necessary for detection of the response can be generated in the transmitter 22 by using delay components (see FIG. 5) and digital I and Q generator (see FIG. 8). The output of each channel is then analyzed in the image construction module 26.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

What is claimed is:

1. A method for conducting spectral scanning magnetic resonance imaging, the method comprising steps of:
establishing a controlled and deterministic inhomogeneous magnetic field in an imaging volume;
simultaneously introducing a plurality of distinct magnetic excitation signals into the imaging volume; and
sensing the response spectrum induced in the imaging volume by the plurality of distinct magnetic excitation signals.

2. The method of claim 1, wherein the distinct magnetic signals comprise different frequencies signals.

3. The method of claim 1, wherein the distinct magnetic signals comprise signals different waveform shapes.

4. The method of claim 1, further comprising repeating said step of simultaneously introducing with a plurality of modified excitation signals over time and repeating said step of sensing to sense the response spectrum induced in the imaging volume by the modified excitation signals over time.

5. The method of claim 1, further comprising repeating said step of simultaneously introducing excitation signals over time and repeating said step of sensing to sense the response spectrum induced in the imaging volume by the excitation signals over time while a target object in the imaging volume is moved over time.

6. The method of claim 1, further comprising a step of analyzing the response spectrum sensed in said step of sensing to extract tomographic information.

7. The method of claim 1, wherein:
said step of establishing a controlled and deterministic inhomogeneous magnetic field is conducted with a magnet;
said step of simultaneously introducing a plurality of distinct magnetic excitation signals into the imaging volume is conducted with a an excitation coil; and
said step of sensing is conducted with a magnetic sensor.

8. The method of claim 7, further comprising using a transmitter that provides signals to the excitation coil, wherein the transmitter comprises:
a digital signal generator that creates multiple distinct frequency signals using a reference frequency;
envelope-shaping amplifiers receiving I and Q phase versions of the multiple distinct frequency signals;
a summer for summing the I and Q phase versions of the multiple distinct frequency signals; and
an amplifier amplifying signals from the summer and providing the signals from the summer to the excitation coil.

9. The method of claim 8, further comprising using a receiver for receiving signals from the magnetic sensor, the receiver comprising:
a low noise amplifier receiving signals from the magnetic sensor;
a mixer that down-converts by I and Q signals from the low noise amplifier for each distinct frequency;
a low pass filter filtering signals from the mixer;
an output amplifier amplifying signals from the low pass filter; and
an analog to digital converter converting the signals from the output amplifier.

10. The method of claim 9, wherein the receiver and transmitter comprises integrated circuits.

12. The method of claim 10, wherein the magnet is sized to make the system portable.

13. A system for conducting spectral scanning magnetic resonance imaging, the system comprising:
means for establishing a controlled and deterministic inhomogeneous magnetic field in an imaging volume;
means for simultaneously introducing a plurality of distinct magnetic excitation signals into the imaging volume; and
means for sensing the response spectrum induced in the imaging volume by the plurality of distinct magnetic excitation signals.
